(19) United States
(12) Patent Application Publication
(10) Pub. No.: US 2014/0244229 A1
(43) Pub. Date: Aug. 28, 2014

(54) Volatile Compound Fingerprint Atlas-Spectrum Model Used for Early Gastric Cancer Diagnosis/ Warning

(71) Applicants: Yixia Zhang, Shanghai (CN); Daxiang Cui, Shanghai (CN)

(72) Inventors: Yixia Zhang, Shanghai (CN); Daxiang Cui, Shanghai (CN)

(21) Appl. No.: 14/119,428

(22) PCT Filed: Jan. 17, 2012
(86) PCT No.: PCT/CN2012/000083

§ 371 Date: Nov. 21, 2013

(30) Foreign Application Priority Data
Nov. 16, 2011 (CN) 201110362943.5

(51) Int. Cl. G06F 19/12; G01N 33/50

(52) U.S. Cl. CPC G06F 19/12; G01N 33/5091
USPC 703/11; 435/29

(57) ABSTRACT

Volatile organic compounds emitted from gastric cancer cell metabolite is separated and detected using HS-SPME/GC-MS. A volatile compounds fingerprint atlas-spectrum model are disclosed for early gastric cancer diagnosis/warning Volatiles 3-octanone and 2-butanone as indicators of gastric cancer cells which are not contained in the headspace of gastric mucosal cells GES-1. Meanwhile, the ratio of mass to volume to concentration of 4-isopropoxy alcohol, nonanoic acid, and 4-butoxy n-butanol is as follows: 4-isopropoxy alcohol [gastric cancer cells]/[normal gastric mucosa cells]≦0.31; nonanoic acid [gastric cancer cells]/[normal gastric mucosa cells]≦0.36; and 4-butoxy n-butanol [gastric cancer cells]/[normal gastric mucosa cells]≦0.40. The volatile organic compound in cell metabolite to be detected is compared with the fingerprint atlas-spectrum model, so as to implement screening and warning of the early gastric cancer.

### CROSS REFERENCE OF RELATED APPLICATION

This is a U.S. National Stage under 35 U.S.C. 371 of the International Application PCT/CN2012/000083, filed Jan. 17, 2012, which claims priority under 35 U.S.C. 119(a-d) to CN 201110362943.5, filed Nov. 16, 2011.

### BACKGROUND OF THE PRESENT INVENTION

1. Field of Invention

The present invention relates to a compound fingerprint atlas-spectrum model used for early gastric cancer diagnosis/warning and a model establishing method, so as to provide help for the warning of gastric cancer.

2. Description of Related Arts

The incidence rate of gastric cancer ranks No. 2 in that of malignant tumor and its case fatality rate ranks No. 1 in China. In addition, the proportion of young patients has been increasing in recent years. Nowadays, gastric cancer is mainly detected through methods based on one or more technologies of X-ray barium meal, fiber endoscopy (including ultrasonic endoscopy), histopathological examination, and serum tumor markers. In the methods mentioned above, radiation received and medicine taken by the patients during the examination will bring a certain side effect, and there is great limitation to the applicable objects. The methods diagnose gastric cancer only based on the tumor size, and have low confirmation rate to early or micro gastric cancer. The cancer is always confirmed in the terminal state, so that the treatment and prognosis of the patients are too late. How to confirm the early gastric cancer is a challenging medical problem. Facts prove that the products of cell metabolism contain a lot of disease markers, while the volatile cell metabolites contain a lot of products which are not found at all and can serve as cancer markers. Due to canceration, the physiological and biochemical conditions of cells have abnormal changes and produce some volatile metabolites, e.g. the oxidative stress of the cells is enhanced during the canceration process, so that the activity of oxygen free radical is enhanced, polyunsaturated fatty acids on the surfaces of cell membranes are oxidized into volatile alkane, aldehyde and other compounds. Therefore, the establishment of the fingerprint atlas-spectrum of gastric cancer cells volatile metabolite may have a certain medical value to the discovery and confirmation of early gastric cancer.

Chromatographic technique has been widely applied to detect the volatile metabolites released from cancer cells in recent years with high detection sensitivity. Solid-phase microextraction (SPME) is a sample enriching and concentrating technology which is environmental-friendly, does not need any solvent, and is convenient and quick. The principle thereof is to select the solid-phase absorption coatings (SPME) made of different nanomaterials according to the polarity difference of the substances, and selectively absorb and concentrate some type of volatile organic targets in a certain system. Currently, the extraction head manufactured by Supelco is widely used. However, the technology is based on the good sample sources. If the volatile metabolites of cells are not well kept before carrying out the solid-phase microextraction, some potential biomarkers may be omitted, and the main reasons lie in that: (1) the concentration of volatile substances from cancer cell metabolite is quite low, and the content thereof is usually trace and even ultra trace; (2) cancer cell metabolite is a dynamic process depending on time, and most volatile markers are intermetabolites of the cells, so the cell culture time is extremely important to the screening of the volatile markers; and (3) the conditions of HS-SPME will directly affect the detection results.

The Chinese patent ZL200410053327.1 provides a protein fingerprint atlas-spectrum model which can be used for liver cancer diagnosis. The model tests the peripheral serum samples of a normal person as well as patients with liver cancer, liver cirrhosis and chronic hepatitis with the protein chip flight time chromatographic system, finds out special protein peaks which are significantly different from thereof liver cancer patients, and works out protein fingerprint atlas-spectrum, including the protein fingerprint atlas-spectrum for liver cancer and liver cirrhosis, liver cancer and chronic hepatitis, liver cancer patients and normal people, and liver cancer and non-liver cancer identification according to the mass-to-charge ratio m/z of all protein peaks and the corresponding protein peak intensity coefficients A. As long as one-by-one comparison and analysis are conducted to m/z and the A value of the corresponding protein in the serum of the subject and those of the fingerprint atlas-spectrum can diagnose initially the liver cancer.

There is no report which is closely related to the test of trace volatile organic substances in the gastric cancer cell metabolite or any fingerprint atlas-spectrum model used for early gastric cancer diagnosis and warning.

### SUMMARY OF THE PRESENT INVENTION

In view of the technical problem, an object of the present invention is to provide a compound fingerprint atlas-spectrum model used for early gastric cancer diagnosis/warning, and the model can be used for the screening and warning of early gastric cancer, so as to provide a new scientific basis for the screening of early gastric cancer.

In view of another technical problem, an object of the present invention is to provide an establishing method of the compound fingerprint atlas-spectrum model used for early gastric cancer diagnosis/warning.

In order to achieve the objects mentioned above, technical scheme adopted by the present invention is as follows.

According to the compound fingerprint atlas-spectrum model used for early gastric cancer diagnosis/warning, a ratio of mass to volume of trace volatile organic compounds 4-isopropoxylbutanol, nonanal and 4-butoxybutano in gastric cancer cell metabolite is separated and detected by using a gas chromatography-mass spectrometer, comparison and statistics are conducted to the ratio of mass to volume thereof and that of a normal gastric mucosal cell, and drawing is performed to form the model according to the comparison results. The ratio of mass to volume to concentration of the 4-isopropoxylbutanol, the nonanal, and the 4-butoxybutano is as follows: 4-isopropoxylbutanol [gastric cancer cells]/[normal gastric mucosa cells]≦0.31; nonanal [gastric cancer cells]/[normal gastric mucosa cells]≦0.36; and 4-butoxybutano [gastric cancer cells]/[normal gastric mucosa cells]≦0.40. The reference value of the substance concentration in the model is that the ratio of mass to volume of the substances in the normal gastric mucosal cell is 100%.

According to the compound fingerprint atlas-spectrum model used for early gastric cancer diagnosis/early-warning, comparison and analysis are conducted to the concentration of the 4-isopropoxylbutanol (Peak 5), the nonanal (Peak 6) and the 4-butoxybutano (Peak 9) in the volatile organic substances of the tested cell metabolite and the fingerprint atlas-spectrum model, and early gastric cancer can be initially warned.

Furthermore, according to the compound fingerprint atlas-spectrum model used for early gastric cancer diagnosis/warning, the volatile organic metabolite has characteristic peaks: 3-octanone (peak 2) and 2-butanone (peak 8). They exist in gastric cancer cells but not in normal cells (0). Therefore, as long as chromatogram can detect substances having the characteristic peaks, the early-warning effect of early gastric cancer can be further supplemented and strengthened.

The model establishing method of the compound fingerprint atlas-spectrum model used for early gastric cancer diagnosis/warning selectively enriches volatile organic substances including alkane, methylation alkane, aldehyde, ketone, alcohol, unsaturated alkane, benzene derivative, halide, etc. in the gastric cancer cell metabolite by culturing cells and optimizing solid-phase microextraction conditions during the sample preparation process through a headspace extraction technology; separates and detects the compounds which are obtained by extraction through the gas chromatography-mass spectrometer, screens volatile organic metabolite related to the gastric cancer cells, and conducts initial qualitative analysis to the detected substances through the gallery NIST08 of the chromatogram; and conducts quantitative analysis to the detected substances, and establishes the “fingerprint atlas-spectrum” model of the volatile compounds of the gastric cancer cells by drawing.

The model establishing method comprises the following steps:

a) collecting culture media of gastric cancer cell MGC-803 and gastric mucosal cell GES-1;

b) volatile metabolites in samples through the headspace solid-phase microextraction technology, wherein extraction head utilized thereof is 75 μm CAR/PDMS, and enrichment time thereof is 45 minutes.

c) separating and detecting substances enriched in step b) through the gas chromatography-mass spectrometer;

d) screening substances in the metabolites of gastric cancer cell MGC-803 and gastric mucosal cell GES-1 with different ratio of mass to volume;

e) drawing to establish a fingerprint atlas-spectrum model based on the comparison and statistics of the ratio of mass to volume of the different substances, wherein the ratio of mass to volume of the 4-isopropoxylbutanol, the nonanal, and the 4-butoxybutano in the fingerprint atlas-spectrum model is as follows: 4-isopropoxylbutanol [gastric cancer cells]/[normal gastric mucosa cells]≦0.31; nonanal [gastric cancer cells]/[normal gastric mucosa cells]≦0.36; and 4-butoxybutano [gastric cancer cells]/[normal gastric mucosa cells]≦0.40, which can be used for the initial screening of early gastric cancer.

The method is simple and safe to operate, the sample to be tested is the cell metabolite which is cultured in vitro. The gastric juice, saliva, urine, etc. of a stomach trouble patient can be also adopted for analysis. The sources of the samples are painless and non-invasive, the resources are rich, so that the method is applicable to people of all ages.

The invention makes up for the defects of the existing early gastric cancer screening technologies, seeks and screens the “fingerprint atlas-spectrum” model of the volatile organic compounds in the gastric cancer cell metabolite for the early-warning of early gastric cancer. The detection rate of the fingerprint atlas-spectrum to the gastric cancer cells reaches 98% in the identification of various cancer cells, including lung cancer, breast cancer, melanoma cancer and gastric cancer cells. Meanwhile, the obtained fingerprint atlas-spectrum also exists in the air exhaled by the gastric cancer patients, and has no significant difference in normal groups of the patients with gastric benign lesions, which will provide a certain bases for the application of the fingerprint atlas-spectrum to the early-warning and screening of the clinical early gastric cancer.

### BRIEF DESCRIPTION OF THE DRAWINGS

Drawing 1 shows the gas chromatogram of metabolites in gastric cancer cells and normal gastric mucosal cells;

Drawing 2 shows the quantitative difference between the metabolites of gastric cancer cells and normal gastric mucosal cells;

Drawing 3 shows the fingerprint atlas-spectrum model according to a first embodiment of the invention.

Drawing 4 is the fingerprint atlas-spectrum model according to a second embodiment of the invention.

### DETAILED DESCRIPTION OF THE PREFERRED EMBODIMENT

Next, the invention is further stated by integrating the preferred embodiments. We shall understand that the embodiments are only used for explaining the invention but not restricting the scope of the invention. The experiment methods of which the specific conditions are not indicated in the following embodiments usually test under conventional conditions or conditions suggested by the manufacturers, and the reagent is specially used for culturing cells.

Reagent and instruments: Improved RPMI-1640 cell culturing medium (Hyclone), new-born calf serum (GIBCO), penicillin-streptomycin, Trypsin-EDTA Solution (Hangzhou Sijiqing), cell culture incubator (Thermo), GC/MS (QP-2010E, Shimadzu), 75 cm² sealed cell culture bottles (Qcbio Science & Technologies Co., Ltd); 57330U manual sample introduction handle, 75 μm CAR/PDMS SPME (SUPELCO);

Humanized gastric cancer cell MGC-803 and gastric mucosal cell GES-1 are from the Cell Bank of Chinese Academy of Sciences.

Experimental steps: The humanized gastric cancer cell MGC-803 and gastric mucosal cell GES-1 which are cultured adherently are passed into the 75 cm³ sealed cell culture bottles by the density of 1*10⁶/mL after trypsinization, centrifugation, collection and blood counting; 40 mL of improved RPMI-1640 cell culture medium containing 5% of new-born calf serum is added in; and bottle caps are tightened, the cells are cultured in 5% of CO₂ for 18 h to 24 h at the constant temperature of 37° C., and the cell vitality is maintained to be about 90%.

6 mL of culture medium in which the gastric cancer cell MGC-803 to grow, 6 mL of culture medium in which the gastric cancer cell GES-1 grows and 6 mL of culture medium in which no cell grows and is cultured under the same conditions are respectively collected and put into the 20 mL top empty bottle.

The samples are respectively extracted and concentrated by HS-SPME (75 μm CAR/PDMS), stirred at the speed of 1200 rpm/min in water bath at 37° C., and extracted for 40 min. Pyrolysis and adsorption are carried out for 2 min at the gas chromatography sample inlets at 280° C., in such a manner that the target molecules are thoroughly desorbed, samples are introduced in the non-shunt mode, the shunt valve is opened 1 min later, and the split ratio is 1:20. Separation is carried out through the capillary-column chromatography Rxi-5 ms (30 m*0.22 mm*0.25 μm). Programmed temperature rise conditions are as follows: the initial temperature 40° C. is maintained for 5 min; and then the temperature rises to 260° C. at the speed of 10° C./min, and maintained for 10 min. The chromatograph scans 42-400 amu in a full range, the electron impact energy is 70 eV, the quadrupole chromatogram ion source temperature is 200° C., the carrier gas is high-purity helium, and the flow rate is 44.2 cm/s. The detected substances are initially qualified by the NIST08 gallery of the chromatogram, and substances with the similarity of above 75% are quantified by relative peak area.

Result:

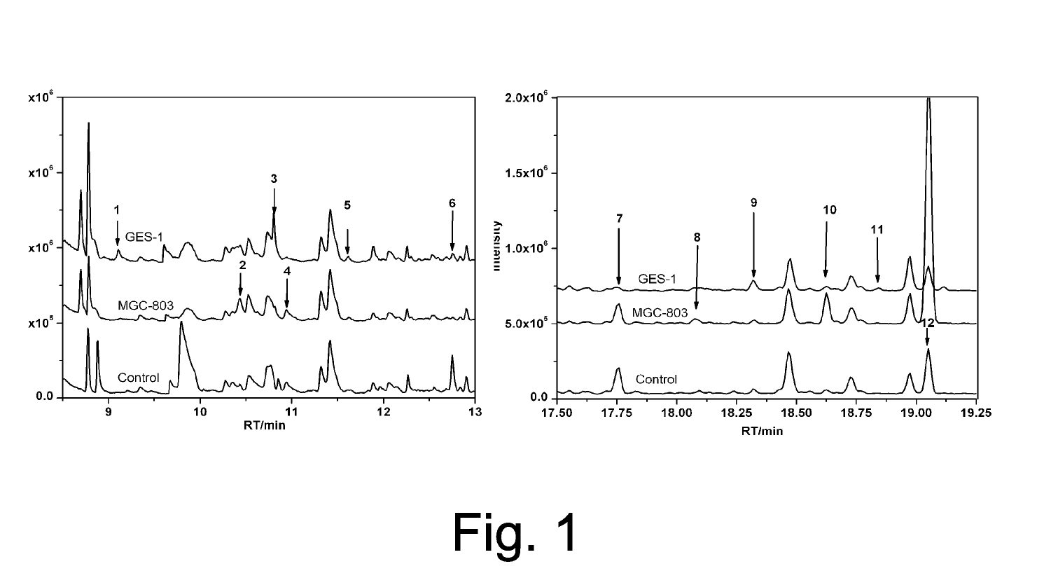

The gas chromatogram of volatile organic substances in gastric mucosal cell strain GES-1, gastric cancer cell strain MGC-803 and the blank culture medium are as shown in FIG. 1. From FIG. 1, we can see that the volatile organic substances in the headspace of GES-1 cell and in the headspace of MGC-803 cell metabolite are qualitative different. The volatile organic metabolite in the headspace of gastric cancer cell MGC-803 has characteristic peaks: 3-octanone(peak 2), 2-butanone(peak 8), peak 10 (substance to be qualified).

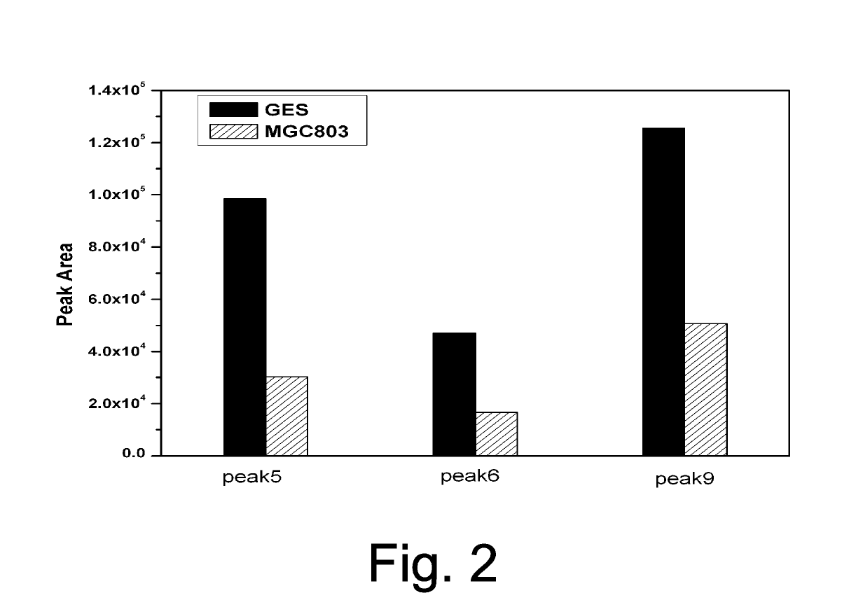

Apart from that, three volatile substances, i.e. 4-isopropoxylbutanol (Peak 5), nonanal (Peak 6) and 4-butoxybutano (Peak 9) were detected both in the headspace of gastric cancer cell MGC-803 and normal gastric mucosal cell GES-1 with different concentration (as shown in FIG. 2). the ratio of concentration is as follows: 4-isopropoxylbutanol[gastric cancer cells]/[normal gastric mucosal cells]≦0.31, nonanal[gastric cancer cells]/[normal gastric mucosal cells]≦0.36, 4-butoxybutano[gastric cancer cells]/[normal gastric mucosal cells]≦0.40. The reference value of the concentration is that the ratio of mass to volume of the substances in the normal gastric mucosal cell is 100%. Generally, the ratio of mass to volume of the three substances in the headspace of normal gastric mucosal cell GES-1 is: 4-isopropoxylbutanol, 0.05%; nonanal, 0.06%; and 4-butoxybutano, 0.23%.

Through the different concentration and features of the volatile substances, the “fingerprint atlas-spectrum” model of the volatile metabolites of the gastric cancer cell is established, which is used for distinguishing gastric cancer cells from normal gastric mucosal cells, and provides new bases for the screening of early gastric cancer.

What needs to be pointed out is that the technicians in the field totally can convert the analysis and experiment critical points of all target molecules identified by relative peak area in the invention into other units by common sense, but not limited to the analysis and experiment critical points identified by ng/ml and pg/ml.

### Embodiment 1

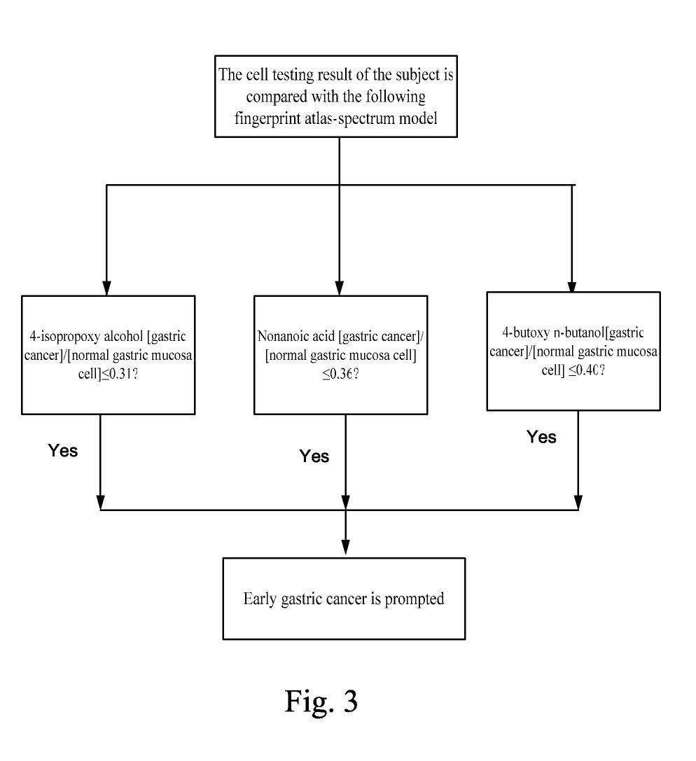

As shown in FIG. 3, the cell to be tested is taken out; the concentration of 4-isopropoxylbutanol (Peak 5), nonanal (Peak 6) and 4-butoxybutano (Peak 9) in the volatile metabolite of the cell is tested; and the test results are compared with the fingerprint atlas-spectrum model of the organic compounds: 4-isopropoxylbutanol[gastric cancer cells]/[normal gastric mucosal cells]≦0.31, nonanal[gastric cancer cells]/[normal gastric mucosal cells]≦0.36, 4-butoxybutano[gastric cancer cells]/[normal gastric mucosal cells]≦0.40, and early gastric cancer is initially screened.

### Embodiment 2

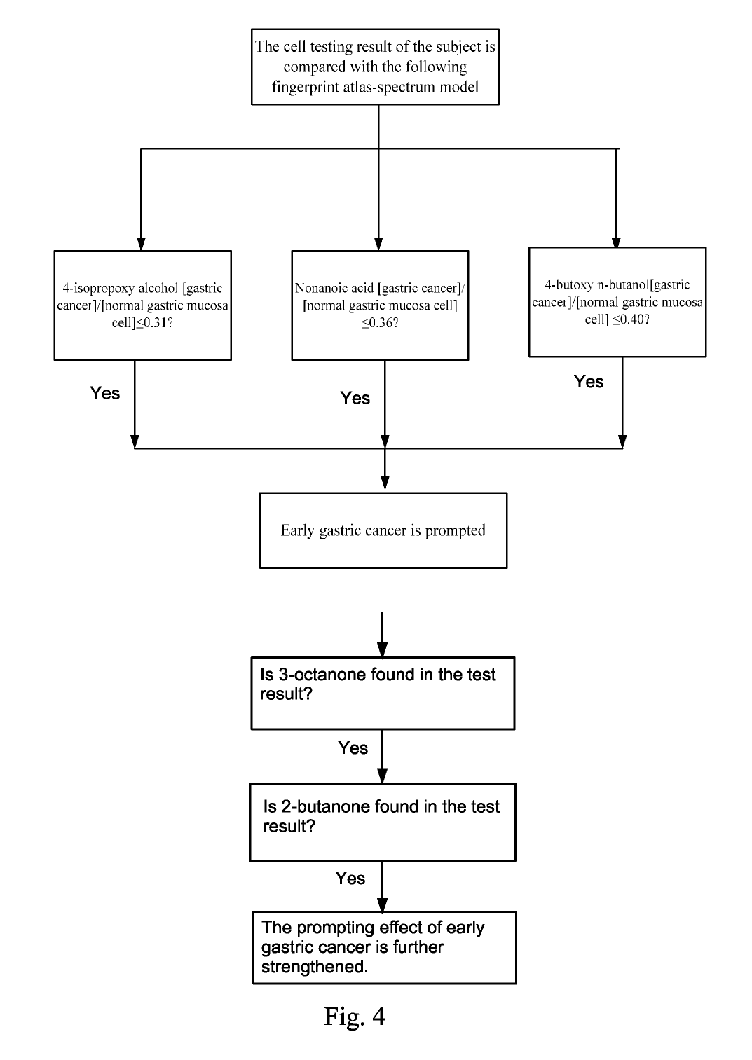

As shown in FIG. 4, the cell to be tested of the subject is prepared; the volatile metabolite in the cell is tested; the test result is compared with the fingerprint atlas-spectrum model of the organic compounds, and analysis is conducted according to the flowchart in the model: 4-isopropoxylbutanol[gastric cancer cells]/[normal gastric mucosal cells]≦0.31, nonanal[gastric cancer cells]/[normal gastric mucosal cells]≦0.36, 4-butoxybutano[gastric cancer cells]/[normal gastric mucosal cells]≦0.40, and early gastric cancer is initially screened. The further test shows that the volatile organic metabolite has characteristic peaks: 3-octanone (peak2) and 2-butanone(peak8). Then the effect of prompting the early gastric cancer can be further strengthened.

According to the invention, the warning of the early gastric cancer is carried out at the cell level through the model. The detection rate of the fingerprint atlas-spectrum to the gastric cancer cells reaches 98% in the testing of various cancer cells, including melanoma cancer, lung cancer, gastric cancer and control group cells.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1-4. (canceled)

5. A compound fingerprint atlas-spectrum model used for early gastric cancer diagnosis/warning, wherein a ratio of mass to volume of trace volatile organic compounds 4-isopropoxylbutanol, nonanal and 4-butoxybutano in gastric cancer cell metabolite is separated and detected by using a gas chromatography-mass spectrometer, comparison and statistics are conducted to the ratio of mass to volume thereof and that of a normal gastric mucosal cell, and a drawing is performed to form the model according to the comparison results, wherein the ratio of mass to volume to concentration of the 4-isopropoxylbutanol, the nonanal, and the 4-butoxybutano is as follows: 4-isopropoxylbutanol [gastric cancer cells]/[normal gastric mucosa cells]≦0.31; nonanal [gastric cancer cells]/[normal gastric mucosa cells]≦0.36; and 4-butoxybutano [gastric cancer cells]/[normal gastric mucosa cells]≦0.40, wherein the reference value of the substance concentration in the model is that the ratio of mass to volume of the substances in the normal gastric mucosal cell is 100%.

6. The compound fingerprint atlas-spectrum model used for early gastric cancer diagnosis/early-warning, as recited in claim 5, wherein in the fingerprint atlas-spectrum model, the volatile organic metabolite further comprises 3-octanone and 2-butanone which are not contained in normal gastric mucosal cells.

7. A model establishing method of the compound fingerprint atlas-spectrum model used for early gastric cancer diagnosis/early-warning, as recited in claim 5, comprising the following steps of:
a) collecting culture media for gastric cancer cell MGC-803 and gastric mucosal cell GES-1;
b) enriching and concentrating volatile metabolites in samples through the headspace solid-phase microextraction technology, wherein extraction head utilized thereof is 75 μm CAR/PDMS, and enrichment time thereof is 45 minutes.
c) separating and detecting substances enriched in step b) through a gas chromatography-mass spectrometer;
d) screening substances in the metabolites of gastric cancer cell MGC-803 and gastric mucosal cell GES-1 with different ratio of mass to volume;
e) drawing to establish a fingerprint atlas-spectrum model based on the comparison and statistics of the ratio of mass to volume of the different substances, wherein the ratio of mass to volume of the 4-isopropoxylbutanol, the nonanal, and the 4-butoxybutano in the fingerprint atlas-spectrum model is as follows: 4-isopropoxylbutanol [gastric cancer cells]/[normal gastric mucosa cells]≦0.31; nonanal [gastric cancer cells]/[normal gastric mucosa cells]≦0.36; and 4-butoxybutano [gastric cancer cells]/[normal gastric mucosa cells]≦0.40, which can be used for the initial screening of early gastric cancer, wherein reference value of the substance concentration in the fingerprint atlas-spectrum model is that the ratio of mass to volume of the substances in the normal gastric mucosal cell is 100%.

8. A model establishing method of the compound fingerprint atlas-spectrum model used for early gastric cancer diagnosis/warning, as recited in claim 6, comprising the following steps:
a) collecting culture media for gastric cancer cell MGC-803 and gastric mucosal cell GES-1;
b) enriching and concentrating volatile metabolites in samples through the headspace solid-phase microextraction technology, wherein extraction head utilized thereof is 75 μm CAR/PDMS, and enrichment time thereof is 45 minutes.
c) separating and detecting substances enriched in step b) through a gas chromatography-mass spectrometer;
d) screening substances in the metabolites of gastric cancer cell MGC-803 and gastric mucosal cell GES-1 with different ratio of mass to volume;
e) drawing to establish a fingerprint atlas-spectrum model based on the comparison and statistics of the ratio of mass to volume of the different substances, wherein the ratio of mass to volume of the 4-isopropoxylbutanol, the nonanal, and the 4-butoxybutano in the fingerprint atlas-spectrum model is as follows: 4-isopropoxylbutanol [gastric cancer cells]/[normal gastric mucosa cells]≦0.31; nonanal [gastric cancer cells]/[normal gastric mucosa cells]≦0.36; and 4-butoxybutano [gastric cancer cells]/[normal gastric mucosa cells]≦0.40, which can be used for the initial screening of early gastric cancer, wherein reference value of the substance concentration in the fingerprint atlas-spectrum model is that the ratio of mass to volume of the substances in the normal gastric mucosal cell is 100%.

9. The model establishing method of the volatile compounds fingerprint atlas-spectrum model used for early gastric cancer diagnosis/warning, as recited in claim 7, wherein the test method is GC/MS, PTR-MS, SIFT-MS or TOF-MS.

10. The model establishing method of the volatile compounds fingerprint atlas-spectrum model used for early gastric cancer diagnosis/warning, as recited in claim 8, wherein the test method is GC-MS, PTR-MS, SIFT-MS or TOF-MS.
